(19) United States
(12) Patent Application Publication
(10) Pub. No.: US 2012/0156635 A1
(43) Pub. Date: Jun. 21, 2012

(54) OPTICAL THERAPEUTIC TREATMENT DEVICE

(75) Inventor: Eric BORNSTEIN, Waltham, MA (US)

(73) Assignee: NOMIR MEDICAL TECHNOLOGIES INC

(21) Appl. No.: 13/190,215

(22) Filed: Jul. 25, 2011

Related U.S. Application Data
(63) Continuation of application No. 12/095,223, filed on Nov. 17, 2008, now abandoned.
(60) Provisional application No. 60/740,776, filed on Nov. 30, 2005.

(51) Int. Cl. A61N 5/067; A61C 17/00

(52) U.S. Cl. 433/29; 433/217.1

(57) ABSTRACT

Methods and devices for Live Biofilm Targeted Thermolysis (LBTT) are disclosed. The disclosed LBTT methods can be used for thermolysis and coagulation of the live periodontal Biofilm with incandescent light and a targeting agent as heat sink. A delivery assembly can be used to deliver the incandescent light generated through the secondary quantum optical and thermal emissions from a carbonized near infrared diode laser delivery fiber, otherwise known as a ‘hot tip,’ to an application region that includes live biofilm. With this novel targeted approach of exploiting the incandescent hot tip's radiant energy (ie. its optical and thermal emissions), the physical nature of the targeted live biofilm in the periodontal pocket is changed from a mucinous liquid-gel, to a semi-solid coagulum, which then facilitates its removal from the effected pocket, with traditional mechanical SRP periodontal techniques.

The present application claims the benefit of related U.S. Provisional Application Ser. No. 60/740,776, filed Nov. 30, 2005, entitled “Optical Therapeutic Treatment Device,” the contents of which are incorporated herein in their entirety by reference.

### FIELD OF THE INVENTION

The present invention relates to methods and devices for selectively reducing the level of a biological contamination in a target site. More particularly, the present invention relates to methods and devices for bacterial decontamination and biofilm elimination in periodontal pockets using optical and thermal radiations.

### BACKGROUND OF THE INVENTION

The term “biofilm” describes a community of microbes enclosed within their own mucinous, gel-like polymer secretions, that are responsible for periodontal and periimplant disease, along with a host of other infectious and inflammatory human ailments.₍₁₎ In periodontal disease, it is the live biofilm that is attached to the dental root and pocket epithelium that protects the pathogenic bacteria from adjunctive treatment modalities such as antibiotics, and endogenous immune functions such as complement activation, chemotaxis of phagocytic cells, and degranulation of polymorphonuclear leukocytes.₍₂₎ These unique protective properties of the biofilm are manifested in part because of the nature of the ecological niche that the bacteria and biofilm live in (in the periodontal pocket), and this secluded location causes the definitive treatment of periodontal disease to be difficult and complex. In fact, typical treatment methods encompassing physical, antimicrobial, and chemical processes for live biofilm elimination are usually necessary.₍₁₎ Two laser therapies that have been previously employed and studied to treat periodontal disease in a non-surgical manner are Laser Sulcular Debridement with an FRP Nd:YAG laser_{(3,4,5)} and Bacterial photo-sensitization with a (soft) Low Level Red Laser and various photo-sensitization agents._{(6,7,8,9,10,)}

Although a number of techniques have been proposed for “bacterial decontamination” in the periodontal pocket with a diverse group of lasers and laser wavelengths, there are scarce references emphasizing the single objective of “periodontal biofilm elimination with lasers” in the literature. Hence, there is a need to explore the inherent thermal properties of inexpensive near infrared diode lasers, as a potential accessory apparatus, to achieve biofilm elimination within the periodontal pocket.

It is accordingly a primary object of the invention to provide methods and devices for biofilm elimination in periodontal pockets.

This is achieved by Live Biofilm Targeted Thermolysis (LBTT) with incandescent light from a “Hot Tip” generated by a CW near infrared diode laser and a targeting agent that selectively absorbs the light energy.

### SUMMARY OF THE INVENTION

The present invention is directed to methods and devices for targeting a live biofilm, thermolysing, and removing that biofilm. In an embodiment of the invention, a targeting substance is introduced to the region containing the biofilm to be thermolysed. The targeting substance is preferably one which is selectively absorbed by the biofilm, such as methylene blue which works for biofilms in periodontal pockets. An optical fiber is provided, where the optical fiber extends between a proximal end and a distal end. The distal end is introduced to tissue near the targeted biofilm, for example, in a periodontal pocket. Then light is introduced into the proximal end of the optical fiber, so that the introduced light propagates toward and exits the distal end of the fiber. The light is preferably coherent, but may be non-coherent and either monochromatic or polychromatic. The intensity of the light is controlled so that upon exit from the distal end of the fiber, sufficient heat is generated to cause tissue and/or fluid near the distal end, to initially carbonize on the distal end, and thereafter cause the carbonized distal end to incandesce. The resulting incandescent radiation is at a wavelength within the preferential absorption spectrum of the targeting substance, so that at least some of the incandescent radiation is absorbed by the targeting substance to a sufficient degree to heat that targeting substance so that the biofilm impregnated with the substance is thermolysed, causing inter alia, the biofilm to form aggregates, which in some forms may be characterized as a semi-solid coagulum. Following thermolysing of the biofilm, the aggregates are removed via periodontal armamentarium followed by for example flushing with a stream of carrier fluid, such as water.

In another embodiment of the invention, an optical therapeutic device is used to deliver the required energy to the treatments area (e.g., MB solution). The optical therapeutic device may comprise one or more components including the various elements required to deliver such optical energy to the MB solution. As one example, the optical therapeutic device may be a hand held device comprising a housing that secures a flexible optical fiber such that the fiber's distal portion is used for generating incandescent light and for treatment.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

For a fuller understanding of methods, and devices of the present disclosure, reference is made to the following detailed description, which is to be taken with the accompanying drawings, wherein:

FIG. 1 illustrates the gum and teeth embedded therein; wherein a diode hot tip is glowing within the periodontal pocket beside one of the teeth.

FIG. 2 is a graphic representation of a diode hot tip emitting first red and then orange visible light as evidenced by a C.I.E. Chromaticity Map overlaid onto FIG. 1.

FIG. 3 is a representation of the heat conversion into electromagnetic energy in the form of incandescence observed with a “red hot” bacterial transfer loop heated to approximately 1000° C. in a traditional Bunsen burner.

FIG. 4 shows an 11 mm deep periodontal pocket on the side of a tooth.

FIG. 5 depicts the application of methylene blue to the periodontal pocket with a fiber brush.

FIG. 6 illustrates the insertion of distal end of the fiber optic within the periodontal pocket and the formation of incandescent tip within the pocket.

FIG. 7 shows the change in location of the incandescent tip within the pocket as a mean to indicate that the incandescent tip can move throughout the pocket in 15-20 seconds.

FIG. 8 depicts the biofilm and tissue coagulum being removed from the irradiated pocket by Gracey scaler.

FIG. 9 illustrates blanching sulcular gingival tissue and presumed new attachment to the tooth 8 days post-op.

FIG. 10 shows the healing and attachment of the gingival tissue to the tooth 5 weeks post op.

FIG. 11 depicts an exemplary handle of a LBTT device connected at one end to a laser source and at another end a light emitting probe.

FIG. 12 depicts an exemplary light emitting probe of a LBTT device having a mating section (1) engaged with the handle, a flex portion (2), and a fiber optic (3) through which the laser propagates to the distal tip for the generation of incandescent light.

### DETAILED DESCRIPTION OF THE DISCLOSURE

In accordance with one aspect of the invention, LBTT is a procedure that specifically targets the live biofilm in the periodontal pocket with a heat sink, for its subsequent thermolysis and facilitated removal. Once the live biofilm is targeted, the inherent radiant emissions from a diode laser generated hot tip are then utilized and exploited, to thermally change the physical nature of biofilm from a liquid-gel to that of a semi-solid coagulum, to make possible its mechanical removal from the periodontal area.

LBTT utilizes a CW near-infrared diode laser, and has fundamentally different dosimetry parameters and logic than either one of the methods known as Laser Sulcular Debridement with an FRP Nd:YAG laser and Bacterial photo-sensitization with a (soft) Low Level Red Laser with various photo-sensitization agents.

Laser Dosimetry management for the Periodontal Pocket: The closest treatment corollary to LBTT with a CW diode laser from a dosimetry perspective is the Laser Sulcular Debridement procedure, traditionally accomplished with the Free Running Pulsed Nd:YAG laser. In 1992, Myers suggested specific dosimetry computations for the periodontal pocket with the Nd:YAG, and his work generated a laser dosimetry table, based on each pocket's individual probing depth.₍₁₁₎ This general principal and quantitative formula was used to generate data with an FRP Nd:YAG laser that led to the first FDA market clearance for “Laser Sulcular Debridement”, with the specific language of “The removal of diseased or inflamed soft tissue in the periodontal pocket to improve clinical indices including gingival index, gingival bleeding index, probe depth, attachment level and tooth mobility”, for the FRP Nd:YAG._{(11, 12)}

Gregg and McArthy₍₁₃₎, took the periodontal pocket laser dosimetry concept further, and cited the first case reports of Sulcular debridement utilizing a computation for “Light Dose” to define the “quantity of laser energy delivered to the treatment site.” See Table 1.

TABLE 1
“Light Dose” calculations for FRP Nd:YAG

| | |
|---|---|
| 1) | Light Dose = Laser energy Delivered to Treatment Site |
| 2) | (Ave Power Watts) * (Duration of Treatment(sec)) = Joules |
| | (Total energy/pocket) |
| 3) | Joules (Total energy/pocket)/(pocket depth (mm)) = Joules/mm |
| | (Pocket Depth pd) |

This novel measure of (Joules/mm pd) was stated by Harris in 2003₍₁₁₎, to be similar to the value of a drug dose in (mg/kg body weight), in that the total light dose would define the concentration of laser energy at the treatment site (the periodontal pocket) much as drug dose defines the concentration of a drug in the tissues. Harris concluded that “light dose” is a useful parameter to provide a uniform measure for comparison across similar studies, with potentially differing laser systems.₍₁₁₎ Most recently, Harris, Gregg, McCarthy et al₍₁₄₎, published a retrospective analysis of the recently FDA approved Laser-assisted New Attachment Procedure (LANAP), where the total light dose delivered per pocket was 10-15 J/mm pd. LANAP is also the procedure reported by Yukna et al₍₁₅₎, with the first histologic evidence of periodontal ligament reattachment and regeneration, in the absence of long junctional epithelium, with a laser procedure. The published primary goal of LANAP is debridement, to remove pocket epithelium and underlying infected tissue within the periodontal pocket completely, and to remove calcified plaque and calculus adherent to the root surface. (Table 2)₍₁₄₎ Finally, Harris has estimated (from reviewing other studies) that a “toxic dose” of light energy with the pulsed Nd:YAG, that would potentially damage root surfaces, would be in the range of 20-60 J/mm pd, and that a different dosimetry needed to be developed that is appropriate to each unique laser modality.₍₁₁₎

TABLE 2
Clinical steps of Patented and FDA approved LANAP Procedure
with a Free Running Pulsed Nd:YAG laser

| | |
|---|---|
| 1) | Chart probe depths |
| 2) | Laser Troughing with short duration pulse (a step-down |
| | approach) in the pocket until epithelial lining debris |
| | ceases to accumulate on fiber tip |
| 3) | Scaling and root-planing with addition of piezo-electric |
| | scaler and hand instruments |
| 4) | Second pass with Nd:Yag laser at longer pulse (635 |
| | micro-seconds) |
| 5) | Gingival tissue compressed to aid in clot formation with |
| | splinting of teeth and occlusal trauma relief as |
| | indicated |

Pulsing abilities of the Nd:YAG and CW Diode Lasers: An FRP Nd:YAG laser is capable of pulse durations in the millionths of a second (10₋₆ sec), that allow for very high peak powers (1-2 thousand watts/pulse) for safe and rapid ablation of sulcular epithelium in a periodontal pocket.₍₁₄₎ Exploiting this laser-tissue interaction, a clinician using a FRP Nd:YAG laser for sulcular debridement has the ability to apply an intense burst of laser energy, for a very short time interval, to the sulcular epithelium in the pocket. This ability will cause quick and precise ablation of the epithelial tissue, as the photobiology of the (10₋₆ sec) interaction keeps the ablation front of the laser tissue reaction, ahead of the thermal front of the laser tissue reaction. A CW or gated diode laser placed in the periodontal pocket does not have the high peak power or microsecond pulse abilities of the FRP Nd:YAG. A CW Diode laser has far longer pulse durations in milliseconds (10₋₃ sec or thousandths of a sec; Table 3), with far less peak power, that will not reach the ablation threshold in soft tissues._{(16,17)} As such a CW Diode laser requires a fundamentally different logic and dosimetric approach for closed (periodontal pocket) procedures, because to a large extent, the output power is converted to heat and radiant energy, from what is known as the “hot tip”._{(16,17)}

TABLE 3
Time Conversion Factors for laser “pulse” Math

1 sec = 1,000 milliseconds (ms) (10³ ms)
1 millisecond ms = 10⁻³ = 1/1000 second = 0.001 sec
0.001 sec = 1 millisecond (ms)
1 sec = 1,000,000 microseconds (μs) (10⁶ μs)
1 microsecond μs = 10⁻⁶ = 1/1,000,000 second = .000001 sec
0.000001 sec = 1 microsecond (μs)
10 microseconds = 1/100,000 second
100 microseconds = 1/10,000 second

With the physical pulse limitations of a CW diode taken as a scientific given, the logic for the “light dose” calculations in the periodontal pocket with a CW diode laser must be substantially altered from the recently published LANAP approach with a FRP Nd:YAG. This important distinction is vital for a clinician to comprehend, because the inherent physical and photo-biological differences between the Diode and the FRP Nd:YAG (ie. Diode “hot tip” Contact Vaporization vs. Nd:YAG Ablation), allow for far smaller margins of error with the diodes, due to the substantial heat production at the incandescent tip.₍₁₇₎ Following Harris's suggestion of requiring a quantitative value for a “light dose” for each unique laser modality₍₁₁₎, one aspect of this invention includes the definition of a new set of parameters, with the implementation of different dosimetric values and logic, explicitly tailored to the CW Diode laser in closed periodontal pocket procedures. These parameters, called Diode Laser Pocket Parameters (DLPP), will exploit the Diode lasers inherent phenomenon of generating an incandescent tip in the periodontal pocket, while also producing a measure of safety against burning and injuring adjacent tissues, with excessive heat, power, and/or treatment time.

Creation of the “Hot Tip” with CW Diode Lasers: The physical changes in quantum emissions and photobiology, that will instantaneously occur when a diode laser fiber (dispensing greater than 500 mW of energy) comes into contact with tissue, and carbonizes the tip of the fiber, have been described in depth.₍₁₇₎ It is given that upon carbonization of a diode laser fiber tip, there is an immediate and profound change in the quantum emissions radiating from the fiber in the form of thermally induced incandescence.

The First Law of Thermodynamics states that energy is neither created nor destroyed, it simply changes form. The example of this law with the CW Diode laser in the periodontal pocket is that the electromagnetic energy of the laser beam is absorbed by the carbonized tip, whereupon it vibrates the molecules in the tip and is converted to heat energy. As the tip instantaneously becomes hotter (above 726° C.), the heat is reconverted into electromagnetic energy in the form incandescence, and the tip then emits radiant visible and infrared light, and is now “red hot”._{(18,19)} This resulting secondary quantum emission of the “hot tip” (incandescence), causes fundamentally different heat transfer and photobiologic events in the periodontal pocket and tissues, than would be seen with the diode lasers primary infrared photons.₍₁₇₎ The photobiology of these changes can be partially explained with the second law of thermodynamics.

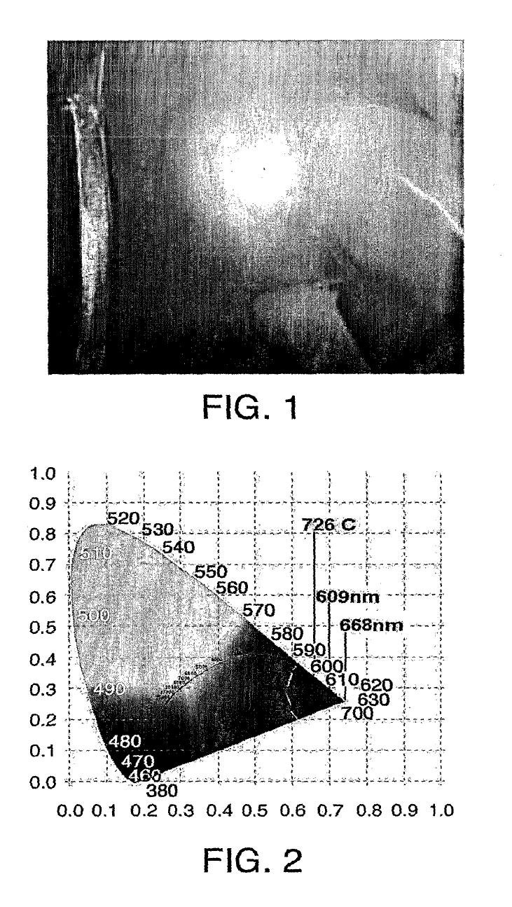

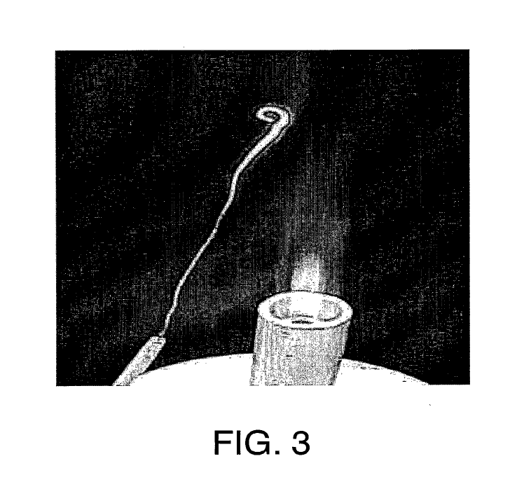

The Second Law of Thermodynamics states that as the primary energy of the laser is converted from one form into another, some of the energy becomes unavailable for further use. This does not mean that some of the laser energy is destroyed, but rather that a portion of the energy in the transfer becomes “waste energy” in a diffuse form (in this example heat) that cannot be used for the same work as the primary photon energy. It can be said that this “heat” or “waste energy” from the hot tip is of a lower quality than the primary photons from the laser, as the lasers primary photons are well collimated, focused and homogeneous, as they emit directly from an uncarbonized fiber._{(19,21)} As the diode hot tip begins to glow with heat (FIG. 1), it emits first red, and then orange visible light. This can be evidenced by a C.I.E. Chromaticity Map overlaid with a black body locus (FIG. 2), as the tip reaches (900 C to 1200 C)₍₁₉₎. Another representation of this energy conversion phenomenon scan be observed with a “red hot” bacterial transfer loop, at approximately 1000 C in a traditional Bunsen burner. (FIG. 3)

With the Hot Tip and degraded fiber optics, the forward beam quality and emissions of primary photons from the laser (measured in terms of energy, focusability and homogeneity) is substantially reduced, and cannot properly continue efficient delivery of high quality energy to the deeper tissues.₍₁₈₎ These quantum changes with “hot tips” and CW diode lasers are real, habitually not understood or taken into account by dental practitioners, and have been previously well described by Verdaasdonk and Swol, and Janda et al._{(20,21)} Hence, the “light dose” computations for the closed pocket FRP Nd:YAG procedures described by Harris, do not reflect the reality of the diode lasers different physics, emissions at the tip, and photobiology.₍₁₁₎

Energy Transfer Differences Between the FRP Nd:YAG and CW Diode Lasers: The traditional Power Density equation for the laser tissue interaction of ablation with the FRP Nd:YAG in the periodontal pocket, measures the potential thermal effect of primary Nd:YAG laser photons at the irradiation area, with a defined beam diameter.

$$Power Density ( W / {cm}^{2} ) = \frac{Laser Output Power ( W )}{Beam Diameter ( {cm}^{2} )}$$

However, with the CW diode laser, the significant amount of the forward emission output power that is converted to local radiant heat at the carbonized fiber tip, greatly damages the fiberoptics and hence eliminates any defined beam area. This heat (from the tip) is then transferred to the proximal periodontal tissues via the mechanism of contact thermal conduction._{(17,18,20,21,22)} Thermal conduction is a fundamentally different mechanism and manner of energy transfer to the tissues than is seen from the FRP Nd:YAG, which is capable of producing adequate “peak” forward power transmission out of the fiber, to achieve tissue ablation. Ablation occurs when the Nd:YAG deposits very high (peak power) energy into a small tissue volume, directly under the delivery tip in millionths of a second. This rapid and contained energy transfer produces the bio-mechanical work of ablation.₍₂₂₎ Hence, the physics of the FRP Nd:YAG allows for most of the laser pulse to be transmitted directly into the tissue under the tip, where the laser energy quickly ablates the tissue, in a far more energy efficient manner than is seen with contact vaporization, via heat conduction, from a hot tip and CW diode laser. Also, the high peak power pulses of the FRP laser most likely assists in the ablation and removal of any debris and detritus caught on the Nd:YAG fiber tip, that would otherwise block the forward laser emission, and build up unwanted heat in the fiber.₍₂₂₎ Conversely, the CW diode is in effect, generating large amounts of incandescent radiating heat energy, in 360 degrees proximal to the tip, as the output power of the laser is largely converted to heat. It is partially for this reason, that there is a greatly altered laser-tissue interaction (thermal contact vaporization vs ablation), seen with the different lasers._{(17, 22)}

Treatment Time—The vital parameter with CW diode lasers in the pocket: To allow for the radiant heat from the incandescent tip when utilizing CW diode laser, the traditional dosimetry equations and logic for closed pocket procedures with the FRP Nd:YAG must be altered, and thought of clinically in terms of Treatment Time, to prevent unwanted tissue damage. For example, Table 2 illustrates that Laser Troughing (in LANAP) should continue in the periodontal pocket with the FRP Nd:YAG laser independent of time, until epithelial lining debris ceases to accumulate on fiber tip. This sulcular debridement procedure is accomplished safely at an average output power of 4 watts and 150 us pulse widths that causes ablation. However, with the incandescent tip, of a CW diode laser, a clinician could only safely use the CW system at a 4 Watt output power for 1-2 seconds, before the proximal periodontal tissues would be irreversibly injured and burned. Hence, this Laser troughing logic and dosimetry for the FRP Nd:YAG cannot be used, and should not be practiced, with the CW diodes, because the 4 Watt the output power will cause a larger amount of energy to be converted to local heat at the fiber tip. The essential Laser Math calculations needed for laser dosimetry with the FRP Nd:YAG and CW diode lasers are shown below and explained in Table 4.

TABLE 4
Laser Math Calculations

```
The Output Power of a laser device, refers to the number of photons
emitted from the laser at a given wavelength and is measured in Watts.
1 (W) = 1000 mW
The Power Density of a laser beam measures the potential thermal effect
of laser photons at the treatment irradiation site/area of tissue. Power
Density is a function of Output Power and Beam Area, is calculated in
(W/cm²), and is the value is obtained with the following equation:
    $\begin{matrix}1 ) Power Density = ( W \text{/} {cm}^{2} ) \\ = \frac{Laser Output Power ( W )}{Beam Diameter ( {cm}^{2} )}\end{matrix}$
The Total Energy delivered into a tissue area by a laser system operating
at a particular output power over a certain period of time, is measured in
Joules, and is obtained with the following equation:
    2) Total Energy (Joules) = Laser Output Power (Watts) × Time (Sec)
It is essential to know the distribution and allocation of the Total Energy
(Joules) delivered into a given tissue area, in order to correctly measure
tissue site dosage for maximal beneficial tissue response. Total energy
distribution will be measured as Energy Density in (Joules/cm²).
The Energy Density is a function of Power Density and Time (sec) seconds,
is measured in (Joules/cm²) and is calculated as follows:
    3) Energy Density (Joules/cm²) = Power Density (Watts) × Time (sec)
Usually, (without a hot tip) to calculate the Treatment Time to deliver a
dose of laser energy to a given volume of tissue, a clinician will need to
know either the Energy Density (J/cm²) or Total Energy (J), as well
as the Output Power (W), and Beam Area (cm²). Treatment time
can then be calculated with the following equation:
    $4 ) Treatment Time ( seconds ) = \frac{Energy Density ( Joules \text{/} {cm}^{2} )}{Power Density ( W \text{/} {cm}^{2} )}$
* However: Because of the “hot tip” phenomenon with diode Laser fibers
in a closed environment (ie. The periodontal pocket), there is no actual
value for “beam area” and hence, there is no practical “Power density”
and/or “Energy Density” equation. Therefore, Treatment Time must rely
on Equation 4a and, for “Light dose” parameters with the CW Diode
Laser within the periodontal pocket.
    $4 a ) Treatment Time ( seconds ) = \frac{Total Energy ( Joules )}{Output Power ( Watts )}$
```

With the direct energy conversion (to heat) of excess output power from the CW diode laser, more heat from the fiber tip would be deleteriously transferred through conduction to the proximal periodontal tissues. From the above (Table 4), it can be seen that by changing ones clinical thought process for the closed (periodontal pocket) procedures with CW diode lasers, and adapting them to a value of Treatment Time (see equation 4a, in table 4), a new “Light Dose” logic can be created with dosimetry parameters that apply to the pocket, based on Time. Furthermore because of the intense heat of the incandescent tip with these lasers, additional clinical modifications to ensure safety, will also involve a lowering of the Total Energy value for a given closed pocket procedure. These specific alterations are necessary for the CW diode laser system in the periodontal pocket, because (as previously described) there is no actual value for “beam area” with the damaged optics of an incandescent hot tip. Without a defined beam area, there can be no practical power density or energy density equation to work with to determine a valid light dose, which is traditionally defined by the primary laser photons delivered to the treatment site directly under an undamaged fiber tip.

Altering the value of total energy to perform safe procedures with the CW diode laser in the periodontal pocket, is simply accomplished by decreasing the laser Output Power (see equation 2, in Table 4) to about ⅓ that of the published Nd;YAG parameters for sulcular debridement procedures such as LANAP₍₁₄₎. These alterations for the CW diode laser will satisfy the requirement of Harris, for developing a new quantitative dosimetry, that is appropriate to each unique laser modality,₍₁₁₎ and will be called Diode Laser Periodontal Parameters (DLPP). This logic for the new CW diode parameters can be easily visualized by a simple restructuring the traditional Total Energy equation in Table 4, to reflect the value of Treatment Time.

Equation restructure for Diode Laser Periodontal Parameters (DLPP)

1) Total Energy (Joules) = Laser Output Power (Watts) × Time (Secs)
[re-order to]
$1 a ) Treatment Time ( sec ) = \frac{Total Energy ( Joules )}{Laser Output Power ( Watts )}$

Utilizing this logic for DLPP, a clinician can simply manipulate both Laser Output Power and/or Treatment Time in a close periodontal procedure, to ensure maximum safety and success with CW diode lasers. Hence, it will be seen with DLPP, that the Output Power that is safe and efficacious for closed intrasulcular Procedures with the FRP Nd:YAG (ave 4 Watts), is approximately a three times (3×) greater output power, than should be safely used with the CW Diodes (1-1.2 Watts). In addition, as the intrasulcular Procedures with the FRP Nd:YAG are performed independent of time (i.e. until epithelial lining debris ceases to accumulate on fiber tip), the CW diode procedures should be completed in approximately 20 to 25 seconds, with rapid tip movement, to prevent unwanted thermal damage to proximal periodontal tissues.

Peak Power—The parameter governing Contact Vaporization vs Ablation: To further quantify the need for DLPP as a guide for new dosimetry parameters with CW diode lasers, the values of peak power with the Nd:YAG and CW Diode, and the ability of peak power to accomplish bio-mechanical work, will be addressed with the computations in Tables 5 and 6.

TABLE 5
FRP Nd:YAG Peak Power calculation for a typical LANAP procedure

Average Output Power (W)/Rep Rate (Hz)/Pulse Duration
(microseconds) = Peak Power/Pulse (W)
Laser Parameters: 150 μs Pulse Duration, at 25 Hz, and 3.9 Watts
Ave. Power
(3.9 W)/(25 Hz)/150 μs (.000150) = Peak Power/Pulse (1040 W/pulse)
Described as a function of Energy per Pulse:
Energy per Pulse = 1040 W/Pulse * 150 μs (.000150) = .156 J/
Pulse or 156 mJ/Pulse
Described as a function of Energy per Sec:
Energy per Sec = .156 J/pulse * 25 pulses/sec * = 3.9 J/sec delivered
to the pocket
Therefore: to obtain Total Energy Delivered to Pocket:
3.9 J/sec * 30 sec treatment time = 117 J delivered to pocket in 30 sec.
Finally: to obtain Energy deliverd in (J mm (pocket depth) pd):
117 J delivered to pocket/8 mm pocket = 14.6 J/mm/pd for a 30 sec
treatment time.

Here (Table 5), it can be seen that for a 30 second application of the FRP Nd:YAG for sulcular debridement, at an average power of 3.9 W and 25 Hz with a pulse duration of 150 μs, the total energy of 14.6 J mm pd, is well within Harris and Gregg's treatment parameters for a safe and effective sulcular debridement procedure like LANAP.₍₁₄₎ With these parameters, the peak power per pulse—or the “power available for the bio-mechanical work of ablation” is 1040 W/pulse for the FRP Nd:YAG laser. However, if a computation is done for a CW diode laser, to understand what the same average power of 3.9 W means in the periodontal pocket with this device, in terms of energy production, and its ability to perform bio-mechanical work, the significant differences between the (laser-tissue interaction) capabilities of the lasers becomes apparent.

TABLE 6
CW Diode Power Calculation for Comparison to FRP Nd:YAG

Laser Parameters: CW Output, for 30 Seconds at 3.9 Watts Ave. Power
To express as Total Energy Delivered to Pocket
3.9 W * 30 Sec = 117 Joules (identical total energy as the FRP Nd:YAG)
Described as a function of Energy per Sec:
117 Joules/30 sec = 3.9 Joules/sec (identical energy/sec as the FRP
Nd:YAG)
BUT Remember:
3.9 W CW is also the “peak power” value for the Diode laser where
(in Table 5)
1040 W/pulse is the “peak power” per pulse- or the “power available for
the work of ablation” for the FRP Nd:YAG, at a pulse duration of
150 μs (.000150 sec)
Therefore the CW Diode laser only has:
3.9 W/1 sec = 3.9 W constant “Peak power” or “Power available for
the work of ablation” in a one second (very long) delivery to the pocket

Hence, as can be seen from this example, the CW diode laser is delivering the same energy to the pocket as the Nd:YAG (3.9 Joules/sec) in one second, but only a “Tissue Ablation Power” of {3.9 W (CW)/1040 W/Pulse*100=0.375%} or one third of one percent!. This (very important calculation) means that the diode is producing 99.625% LESS “Power available for the bio-mechanical work of ablation” of each pulse of the FRP Nd:YAG, in the same one second time interval, with the same energy. This notable computation is correct, as a consequence of the two fundamental definitions of Energy and Power, when the different lasers abilities are linked to the ablation concept of bio-mechanical work. See Table 7.

TABLE 7
Fundamental Definitions of Energy and Power

Energy is defined as the ability to do work.
Power is defined as the rate of doing work, or Power can be used to
describe the amount of work accomplished in a certain period of time.
As an equation this concept is stated:
$Power ( Watts ) = \frac{Work}{Time}$

The computations in Tables 5 and 6 make clear that the CW Diode Laser has the theoretical ability to perform the same bio-mechanical work (ablation) as the FRP Nd:YAG, because both laser systems produce 117 Joules of energy in 30 seconds. However, the critical factor to examine is the function of Power, because the FRP Nd:YAG laser puts out far more power per unit time (99.625% more peak power, at a far faster rate, on the order of 10₋₆ sec), than the Diode. Therefore, because Power is defined as the rate of doing work,₍₂₂₎ the CW diode laser does not produce enough forward emission power per unit time, to cause ablation. Furthermore, remember from the previous discussion of the second law of thermodynamics, that a large portion of the available energy from the CW diode laser is converted to “waste energy” in the diffuse form of heat. Finally, one must also continually keep in mind, there is far less time that the incandescent fiber can stay safely in a closed pocket (at the same energy as the FRP Nd:YAG), for the sulcular debridement parameters because of the conversion to heat.

The last vital issue to comprehend, is that if the CW diode is “pulsed” or “gated”, it actually delivers less total power and hence less energy to the pocket, with no peak power increase (like the FRP:Nd:YAG) for treatment. This translates into even less of a “theoretical ability” to do the bio-mechanical work of tissue ablation. Hence, the most straightforward calculations and heat transfer assessments, in the closed periodontal pocket with these diode devices, come from laser in CW mode,₍₂₂₎ with DLPP.

Even with the above recommended adjustments for CW diode lasers coupled to the new logic of DLPP, any excess time in a closed periodontal procedure (even with max 1.2 W output power) can induce heat related deleterious effects to the periodontal tissues proximal to the incandescent. It is for this reason that the concept of a “heat sink”, to preferentially absorb the incandescent heat energy for the diode laser's hot tip is potentially useful, for not only protecting deeper periodontal tissues from damage, but also for targeting live biofilms in the periodontal pocket for thermolysis with CW diodes.

Live Biofilm Targeting with Methylene Blue (MB): MB has been used previously in medicine as an oxidation reduction indicator, an antidote to cyanide, and as a mild antiseptic. In dentistry, MB has been used primarily as a photo-sensitizer for individual bacteria within the periodontal pocket, and activated with (soft) Low Level Visible Red lasers (Laser output power of 100 mW or less). These applications with low level red lasers have met with little practical in vivo success in the last 10 years in the periodontal pocket._{(6,7,8,9,10,)} The visible soft red lasers that have been generally employed for “photosensitization” of selected periodontopathic bacteria do not generate enough output power for creation of an incandescent tip, and a review of the literature to this effect can be appraised in Table 8.

TABLE 8
Prior methods and techniques of Methylene Blue addition to the Periodontal Pocket

| Type of Study | Laser/Power Used | Result | Reference |
|---|---|---|---|
| In vivo | none | Statistically significant | Wilson et al |
| Sub-gingival | | decrease in Gm-anaerobes, | (1992) (6) |
| MB application | | spirochetes, motile bacteria | |
| 14 days | | | |
| In vitro MB and | 7.3 mW Output | Killing ability detected after, | Dobson and Wilson |
| TB addition to | Power HeNe Soft | 30 sec for Streptococcus sanguis, | (1992) (7) |
| Agar plates of | Red Laser | Porphyromonas gingivalis, | |
| Bacteria | | Fusobacterium nucleatum | |
| | | A.. actinomycetemcomitans | |
| In Vitro cultures of | 7.3 mW Output | MB and TB are effective | Wilson, Dobson |
| S. sanguis P. gingivalis | Soft Red laser | photosensitizers in vitro | Sakar (1993) (9) |
| F. nucleatum, | up to 80 sec | | |
| A.. actinomycetemcomitans | | | |
| Treated with MB or TB | | | |
| At 25 microgramg/ml | | | |
| In vivo Sub-g application | none | sites treated with MB and | Ower et al |
| of MB in slow release | | subgigival debridement at | (1995) (23) |
| device with subgingival | | 56 days showed marginal | |
| debridement on day one | | improvements in pocket depth | |
| | | better than debridement alone | |
| In vivo Supra-g irrigation | Gallium-Arsenide | no additional microbiological | Yilmaz et al |
| of MB as a photosensitizer | 685 nm at 30 mW | benefit was found over | (2002) (24) |
| compared to SRP or with SRP | for 70 sec | conventional mechanical | |
| | | debridement | |
| Testing of six commercially | HeNe soft laser | 1,9-dimethyl Methylene Blue | O'Neill et al |
| Available photosensitisers | at 632.8 nm | achieved complete bacterial kill | (2003) (25) |
| For photobacterial activity | | of Streptococcus sanguis | |
| Against periodontal pathogens | | | |
| Testing if periodontal | 632.8 nm laser at | at an energy density of (21.2 Jj/cm−2) | Chan et al |
| Photodynamic therapy (PDT) | 30 mW output and | the 665 nm laser's primary photons | (2003) (26) |
| Are either wavelength or | 665 nm laser at 100 mW | had far greater bactericidal effect than | |
| Dose dependent in the | 830 nm laser at 100 mW | the primary photons of the 830 nm laser | |
| Presence of MB | | | |

500 mw of Output Power are generally needed to produce a “hot tip” reaction with CW Diode lasers

At first glance, it would seem incongruous to use an infrared laser as an instrument in combination with anything that is stained with MB, as the primary spectral emissions of any infrared lasers start at 150 nm longer than the traditional MB absorption spectra.₍₂₇₎ However, with Live Biofilm Targeted Thermolysis (LBTT), the MB is a biofilm targeting agent and a “heat sink”, for the secondary incandescent (visible) “hot tip” radiant energy generated from the fiber in the pocket. The orange and red visible emissions from the incandescent tip (600 nm-700 nm) are what will be exploited within the MB's traditional absorption curve. This is easily accomplished, as MB has absorption peaks at 609 nm and 668 nm₍₂₈₎ in the visible orange and red spectrum, exactly within the area of the C.I.E. Chromaticity Map (overlaid with a black body locus} for the incandescent temperatures of the hot tip. (FIG. 2)

Biofilms consist of a matrix formed from exopolysaccharide (EPS), water and microbes in percentages of roughly 5% (EPS), 92% (water) and 3% (microbes)_{(1, 30)}. The EPS component is an extremely hydrated gel-like (mucinous) bio-polymer that creates the 3-dimensional structure of the biofilm. It is the EPS matrix that protects the microbes within the biofilm from attack by harmful antimicrobial agents (antibiotics) and the immune system._{(1, 2, 30)} Listgarten et al, has shown that biofilms and diseased epithelium in areas with subgingival dental plaque (biofilm) are highly permeable to MB._{(31, 32, 33)} This is exactly the targeting mechanism of the LBTT procedure. The logic is to target the biofilm (where the bacteria live) with a heat sink (MB) for thermolysis.

Given the above targeting mechanism of the biofilm, it then follows that the intense energy of the photons from the incandescent fiber tip are absorbed by MB molecules impregnating the biofilm, and are then immediately converted to vibrational and rotational energy within the MB molecules, which is the molecular basis for heat. This heat will always raise the temperature of the MB or anything that is stained with MB.₍₂₉₎ Accordingly, by means of this method, with the absorption of secondary incandescent energy from the diode hot tip, there is a profound energy transfer to the live biofilm and diseased sulcular epithelium that has been stained with MB. This novel targeted and controlled heat transfer to the live biofilm, then produces a semi-solid coagulum from the biofilm and stained diseased epithelium, that can them easily be removed with traditional root planning and scaling procedures.

If one thinks of the physical character (not composition) of a biofilm as potentially similar to a raw egg white, the above mechanism and logic for thermolysis and coagulation will become clear. If one were to attempt the removal of raw egg white (biofilm), from a ceramic tile floor (root surface) with a steel spoon (periodontal scaler), it would be virtually impossible to remove the entire gel-like biofilm matrix in its raw gel-like form. However, if the raw egg white was selectively targeted and heated, it would change its physical character to that of a solid coagulum (cooked egg) and hence, be far easier to remove from the tile floor. This is the logic and design of Live Biofilm Targeting in the periodontal pocket with MB and/or other targeting agents used as a heat sink. With this method, a practitioner can comfortably turn down the output power of the diode laser following the DLPP outline, to approximately 1.0 W CW, and accomplish a live biofilm phase change through coagulation and thermolysis of the gel-like matrix. This will lead to a safer procedure for the dental patient, and preserve more collagen, bone, and mucosa in the periodontal pocket from irreversible thermal damage during the procedure, while at the same time, facilitating the removal of the biofilm. Once the biofilm is removed, the pocket has the immediate potential to heal. Through healing the pocket, the body removes the unique ecological niche that fostered the microbial growth, the 8-10 mm subgingival habitat of the periodontal pocket.

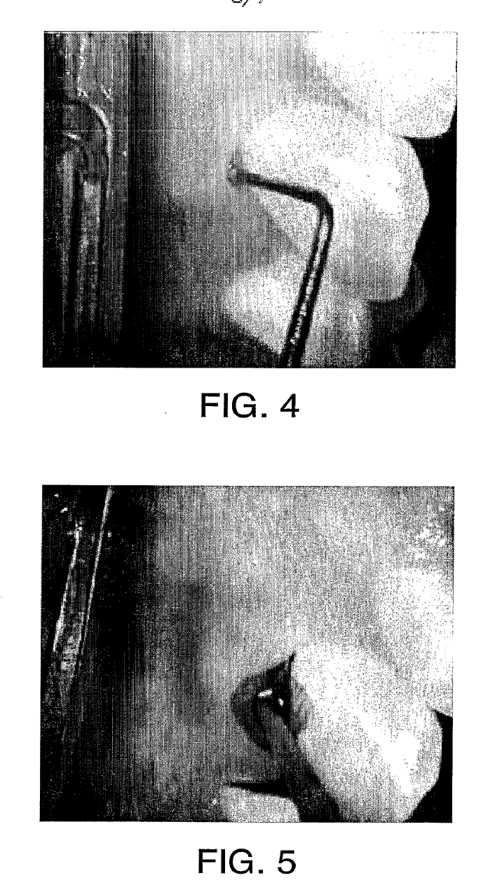

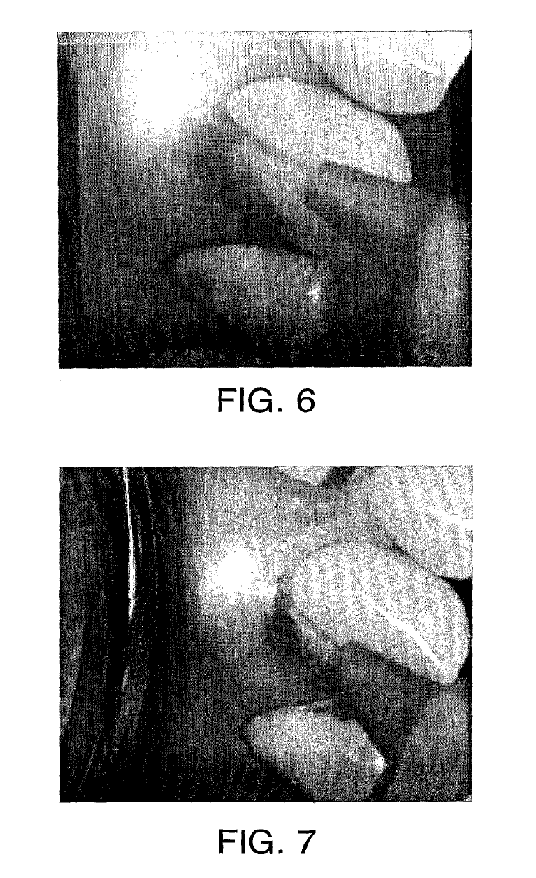

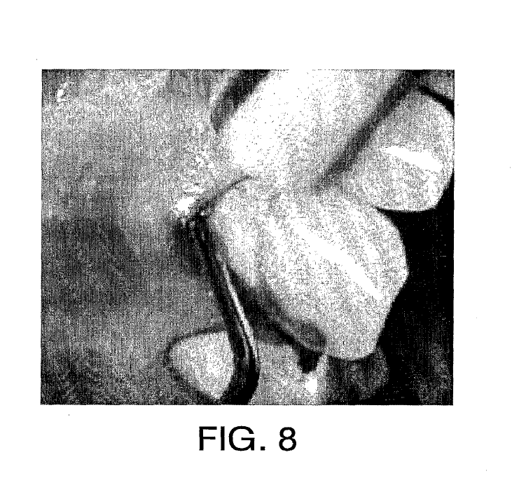

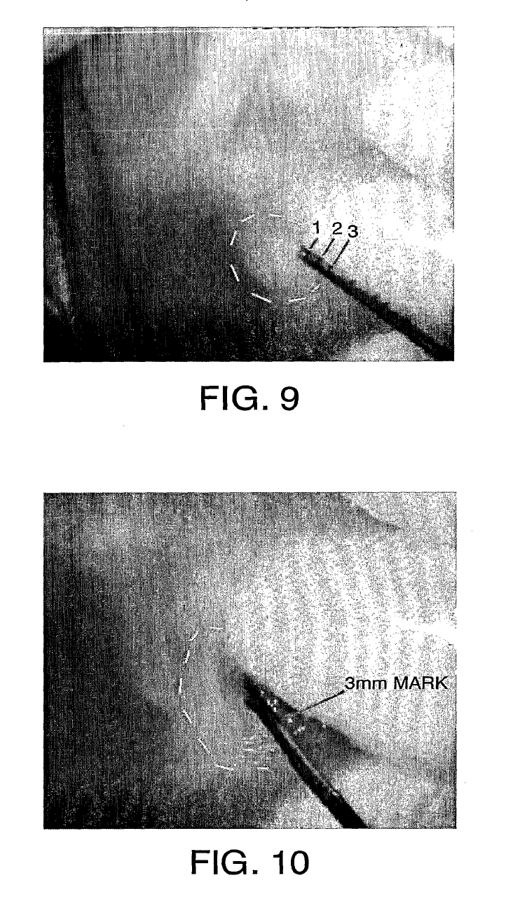

In an exemplary embodiment, Live Biofilm Targeted Thermolysis begins with the introduction of a 1% Methylene Blue solution to the periodontal pocket, delivered with a small fiber brush, to allow access and coverage of the entire 3-dimensional area of the pocket, with the biofilm targeting solution. (FIGS. 4-5) The MB solution is then left in the pocket for one minute, with gentle irrigation of the pocket afterwards, to remove excess solution from the area. A near-infrared diode laser fiber is then placed in the pocket with the output power set to 1 Watt CW and the laser is turned on. Within one second, an incandescent tip is generated (via the mechanisms previously described) and the tip is then rapidly moved throughout the entire area of the periodontal pocket, to commence coagulation of the targeted biofilm and diseased epithelial tissue. (FIGS. 6-7) After 20 seconds of rapid fiber movement throughout the periodontal pocket, the fiber is removed, and a periodontal scaler is introduced to remove the biofilm and tissue coagulum, along with any calculus or other debris from the entire pocket area. (FIG. 8) The pocket is then irrigated with sterile saline through a thin flexible canula and an irrigation syringe, followed by firm pressure of the tissue against the tooth for 2 minutes with moist gauze. The patient was then given 400 mg ibuprophen chair side, and released with instructions to avoid the LBTT area for three days, and then resume normal hygiene. In this case, eight days later, the area of the periodontal pocket could not be accessed with a periodontal probe, as the coronal tissue blanched, signifying new attachment to the root surface by what is assumed to be long junctional epithelium. (FIG. 9) Five weeks post-op, the area was again assessed, with equivalent results. There appeared to be new attachment, with a 2 mm healthy sulcus, and a resolution of the periodontal pocket. (FIG. 10)

In any of the embodiments described herein, or equivalents not specifically disclosed herein, an optical therapeutic device is used to deliver the required energy to the treatments are (e.g., MB solution). The optical therapeutic device may be comprised of one or more components including the various elements required to deliver such optical energy to the MB solution. As one example, the optical therapeutic device may be a hand held device comprising a housing that secures a flexible optical fiber such that the fiber's distal portion for treatment.

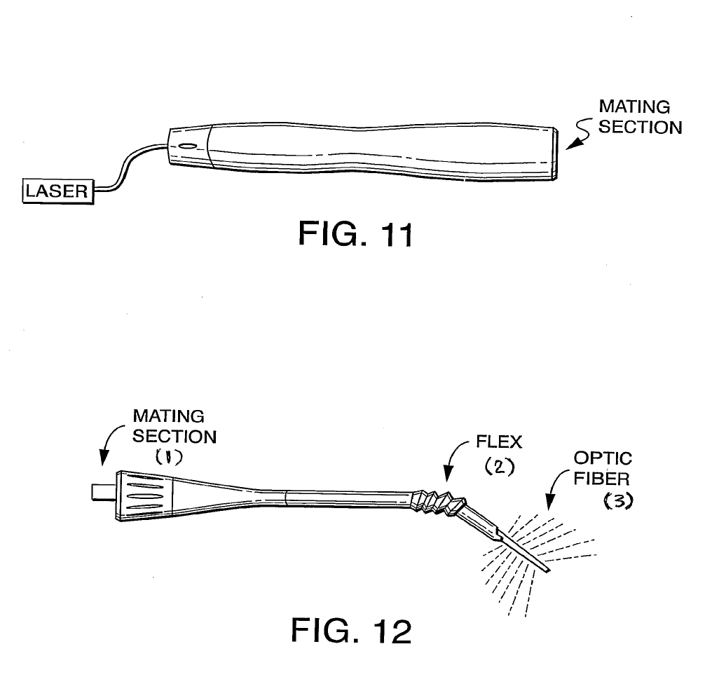

In an exemplary embodiment, the optical therapeutic device of the present disclosure may comprise substantially two components: (1) a handle and a (2) light emitting probe, as shown in FIGS. 11 & 12. In such an embodiment, the handle may be made of, for example, a molded plastic or the like, and may include a system for accepting energy and directing light energy into the optical fiber of the light emitting probe. For instance, a lens system may be included within the handle to channel the light into the mating portion of the light emitting probe when engaged with the mating portion of the handle. The light emitting probe may be configured to be disposable, and the handle portion may be reusable. The probe may include at least one flex region, or may be sufficient pliable or flixible in one or more regions such that it achieves the benefits of the at least one flex portion. The probe may be made of any of a variety of materials, including plastics and the like. Such flexibility allows the probe and optical fiber to be easily and comfortably position in and about the treatment area. The optical fiber may be configured to deliver energy along its lateral portions, for example by employing a Bragg grated fiber used for energy dispersion (see WO 2005/034790), in addition to or as an alternative to delivering light energy from its distal end. Such configurations may be determined based on the specific application for which it is to be used.

The procedure and logic presented has been an exemplary embodiment of Live Biofilm Targeting and Thermolysis application in a periodontal pocket with a near infrared CW diode laser, and 1% MB as a heat sink. Coupled to this procedure is a new computational logic for safer intrasulcular dosimetry with CW diode lasers and the incandescent tip phenomenon in the closed pocket environment. It is vital for a practitioner to understand the differences between CW diodes and FRP Nd:YAG lasers, as the predominance of the laser-periodontal literature has dealt with the FRP Nd:YAG laser and Sulcular Debridement procedures such as LANAP. The published LANAP protocols and dosimetry cannot and should not be followed with CW diode lasers in the closed environment of the periodontal pocket, as the physics and photobiology of the two systems is profoundly different, as was presented here within mathematically. These profound differences will cause entirely dissimilar laser-tissue reactions in the periodontal pocket. LBTT is an attempt to exploit the physical phenomena associated with the CW diode laser of the incandescent tip, by targeting and attacking the live biofilm for thermolysis and removal. DLPP is a new set of dosimetry parameters to attempt to make the incandescent tip useful, and safer, for closed periodontal procedures with the CW diode lasers.

Other embodiments of the invention will be apparent to those skilled in the art from consideration of the specification and practice of the invention disclosed herein. While laser power outputs of 0.5 Watts and 1-1.2 Watts are disclosed in the specification, Applicant believes that a laser with power output of 0.5 to 2 Watts can be safely used in methods and devices of the invention. It is intended that the specification and examples be considered as exemplary only, with a true scope and spirit of the invention being indicated by the following claims.

### REFERENCES

1) Socransky, S. and Haffajee, A. Dental Biofilms: difficult therapeutic targets, Periodontology 2000, Vol 28, 2002, 12-55.
2) Wilson, M. Bacterial Biofilms and Human Disease, Science Progress, 2001, 84 (3), 235-254.
3). White, J M, H E Goodis, C L Rose. Use of the Pulsed Nd:YAG laser for intraoral Soft Tissue Surgery. Lasers Surg Med; 1991; 11: 455-461.
4) Greenwell H, D M Harris, K Pickman, et al, Clinical evaluation of Nd:YAG laser curettage on periodontitis and periodontal pathogens. J Dent Res 1999; 78:138.
5) Gregg R H, McCarthy D K. Laser ENAP for periodontal ligament regeneration. Dentistry Today, 1998; 17:86-89.
6) Wilson et al, A preliminary evaluation of the use of a redox agent in the treatment of chronic periodontitis, J Periodontal Res, 1992, September; 27 (5): 522-7.
7) Dobson and Wilson, Sensitization of oral bacteria in biofilms to killing by light from a low-power laser, Arch Oral Biol. 1992 November; 37(11):883-7.
8) Sarkar and Wilson Lethal photosensitization of bacteria in subgingiva plaque from patients with chronic periodontitis. J Periodontal Res. 1993 May; 28(3):204-10.
9) Wilson, Dobson and Sakar, Sensitization of periodontopathogenic bacteria to killing by light from a low-power laser, Oral Microbiol Immunol. 1993 June; 8(3):182-7.
10) Wilson et at Bacteria in supragingival plaque samples can be killed by low-power laser light in the presence of a photosensitizer, J Appl Bacteriol. 1995 May; 78(5): 569-74
11) Harris, D. Letter to the editor: Dosimetry for Laser Sulcular Debridement, Laser Surg Med 2003; 33: 217-218.
12) Neill M E, J T Mellonig, Clinical efficacy of the Nd;YAG Laser for Combination Periodontitis Therapy. Pract Periodontics Aesthet Dent, 1997; 9 (6 Suppl):1-5
13) Gregg R H, McCarthy D K. Laser Periodontal Therapy: Caser reports. Dentistry Today 2001; 20:74-81.
14) Harris, D., Gregg, R H., McCarthy D K. et al., Laser-assisted new attachment procedure in private practice, General Dentistry, September-October 2004, Vol 52 No 5, pp 396-403.
15) Yukna R A, Evans G H, at al, Human periodontal regeneration laser-assisted new attachment procedure. International Association Dental research 2003; Abstract No. 2411.
16) ALD (The Academy of Laser Dentistry). Featured wavelength: diode—the diode Laser in dentistry (Academy report) Wavelengths 2000: 8: 13.
17) Bornstein E, Near-infrared dental diode lasers. Scientific and photobiologic principles and applications, Dent Today. 2004 March; 23(3):102-8
18) Grant S A, Soufiane A, Shirk G, et al. Degradation-induced transmission losses in silica optical fibers. Lasers Surg Med. 1997; 21:65-71
19) Kuhn T S. Black Body Theoryand the Quantum Discontinuity: 1894-1912.Chicago, Ill.: University of Chicago press; 1978.
20) Verdaasdonk R M, van Swol C F, Laser light delivery systems for medical applications. Phys Med Biol. 1997; 42:869-894.
21) Janda P, Sroka R, Mundweil B, et al. Comparison of thermal tissue effects induced by contact application of fiber guided laser systems. Lasers Surg Med. 2003; 33:93-101.
22) Manni, J. Dental Applications of Advanced Lasers. Burlington, Mass. JGM Associates, Inc. 2000.
23) Ower et al, The effects on chronic periodontitis of a subgingivally-placed redox agent in a slow release device. J Clin Periodontol. 1995 June; 22(6):494-500.
24) Yilmaz et al, Effect of gallium arsenide diode laser on human periodontal disease: a microbiological and clinical study, Lasers Surg Med. 2002; 30(1):60-6.
25) O'Neill J et at, Comparative antistreptococcal activity of photobactericidal agents, J Chemother. 2003 August; 15(4):329-34.
26) Chan et al, Bactericidal effects of different laser wavelengths on periodontopathic germs in photodynamic therapy, Lasers Med Sci. 2003; 18(1):51-5.
27) Niemz M H. Laser-Tissue interactions: Fundamentals and Applications. Berlin, Germany: Springer Verlag; 2002.
28) Atherton, S. J. and Harriman, A. J. American Chemical Society, 1993, 115, 1816-1822.
29) Hewitt P, Conceptual Physics Ninth Edition. San Francisco, USA, Addison Wesley; 2002.
30) Jass, J., Surman, S., Walker, J. Medical Biofilms: Detection, Prevention, and Control. West Sussex, England, John Wiley&Sons. LTD, 2003.
31) Listgarten M A, Formation of dental plaque and other oral biofilms., In: Newman H N, Wilson M ed. Dental Plaque revisited. Cardiff: Bioline, 1999: 187-210.
32) Listgarten M A, Structure of the microbial flora associated with periodontal health and disease in man. A light and electron microscopic study. J Periodontol 1976: 47: 1-18.
33) Listgarten M A, Mayo H E, Tremblay R., Development of dental plaque on epoxy resin crowns in man. A light and electron microscopic study. J Periodontol 1975: 46: 10-26.

What is claimed is:

1. A method of removing a periodontal biofilm in a subject, comprising:
administering to a treatment area an effective amount of a targeting agent that selectively absorbs radiation energy and irradiating the treatment area with incandescent light generated by a near-infrared diode laser source; wherein the light is absorbed by the targeting agent; thereby causing thermolysis and elimination of microbes from the treatment area.

2. A method of eliminating a periodontal biofilm in a subject, comprising: administering to the biofilm an effective amount of a targeting agent and irradiating the biofilm with incandescent light generated by a near-infrared diode laser source; wherein the light is selectively absorbed by the targeting agent, thereby inducing the thermal alteration of the biofilm into a coagulum to facilitate its removal.

3. A method as in claim 2 wherein the biofilm is in a periodontal pocket, a peri implant, or a root canal.

4. A method as in claim 1 wherein the targeting agent is 1% methylene blue.

5. A method as in claim 1 wherein the near-infrared diode laser is a CW laser.

6. A method as in claim 1 wherein the near-infrared diode laser is a pulsed laser.

7. A method as in claim 1 wherein the near-infrared diode laser has an output power between 0.5-2 Watts.

8. An optical therapeutic device for elimination of a periodontal biofilm, comprising: a handle and a light emitting probe housing an optical fiber; wherein the optical fiber delivers near infrared diode laser energy to generate incandescent light at its tip in and about a treatment area.

9. An optical therapeutic device of claim 8 wherein the handle is reusable.

10. An optical therapeutic device of claim 8 wherein the probe is reusable.

11. An optical therapeutic device of claim 8 wherein the probe is disposable.

12. An optical therapeutic device of claim 8 wherein the probe includes at least one flexible portion to allow positioning of the optical fiber tip in and about a treatment area.

13. An optical therapeutic device of claim 8 wherein the optical fiber is configured to deliver energy along its lateral portions.

14. An optical therapeutic device of claim 8 wherein the treatment area is a periodontal pocket, a peri implant, or a root canal.

15. An optical therapeutic device of claim 8 wherein the near-infrared diode laser is a CW laser.

16. An optical therapeutic device of claim 8 wherein the near-infrared diode laser is a pulsed laser.

17. An optical therapeutic device of claim 8 wherein the near-infrared diode laser has an output power between 0.5-2 Watts.

18. A kit for the elimination of a periodontal biofilm comprising:
(a) a optical therapeutic device including:
an optical source including means for generating near-infrared laser; and
a laser delivery apparatus including a handle and a light emitting probe housing an optical fiber; wherein the optical fiber is used for generating incandescent light at a treatment area, said fiber including:
a proximal tip affixed to said optical source;
a distal tip at the end of said fiber opposite said proximal tip, wherein the distal tip is positioned in and about the treatment area, and wherein the laser is converted to incandescent light at the distal tip;
means for coupling said optical source to said optical fiber, and
(b) a targeting agent that is administered to the treatment area; wherein the agent selectively absorbs light energy generated by the optical source; thereby inducing thermolysis.
